(19) United States
(12) Patent Application Publication
(10) Pub. No.: US 2006/0160770 A1
(43) Pub. Date: Jul. 20, 2006

(54) Method for the synthesis of 3-substituted indolizine and benzoindolizine compounds

(76) Inventors: Anthony Hayford, Greenville, NC (US); Joseph Kaloko, Greenville, NC (US)

Correspondence Address: MYERS BIGEL SIBLEY & SAJOVEC, PO BOX 37428, RALEIGH, NC, 27627, US

(21) Appl. No.: 11/319,907

(22) Filed: Dec. 28, 2005

Related U.S. Application Data
(60) Provisional application No. 60/640,369, filed on Dec. 30, 2004.

(51) Int. Cl. A61K 31/724; A61K 31/695; A61K 31/53; A61K 31/519; A61K 31/4745; C07D 487/02; C07D 471/02

(52) U.S. Cl. 514/63; 514/243; 514/259.1; 514/306; 544/184; 544/281; 546/113; 514/58; 536/41; 546/14

(57) ABSTRACT

A method of making a compound of Formula I: 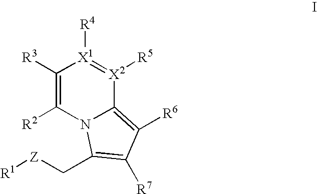
comprises reacting a compound of Formula II 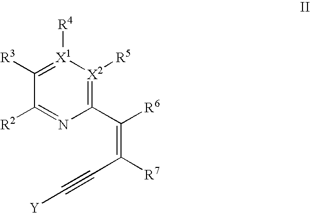
with a compound such as R¹OH or R¹SH, to produce said compound of Formula I. Compounds of Formula I are useful, among other things, as dyes, spectral sensitizers, glycosidase inhibitors, and as antibacterial, antiviral, and anti-inflammatory agents.

### RELATED APPLICATIONS

This application claims the benefit of U.S. Provisional Patent Application Ser. No. 60/640,369, filed Dec. 30, 2004, the disclosure of which is incorporated by reference herein in its entirety.

### FIELD OF THE INVENTION

The present invention concerns methods and intermediates useful for the synthesis of 3-substituted indolizine and benzoindolizine compounds.

### BACKGROUND OF THE INVENTION

Fully or partially unsaturated indolizines have received attention in the literature because of the interesting similarities and diversions in structure to indole.^{1,7} Like indole, indolizine is a 10 π-heteroaromatic ring system. Although isoelectronic with indole, indolizine is an N-bridgehead heterocyclic with both a π-excessive pyrrole and a π-deficient pyridine fused in a bicyclic ring system. Recently, several synthetic efforts have been focused on indolizine ring systems to access their pharmacological activities because of the association with the numerous biologically important indole compounds.^{1,4}

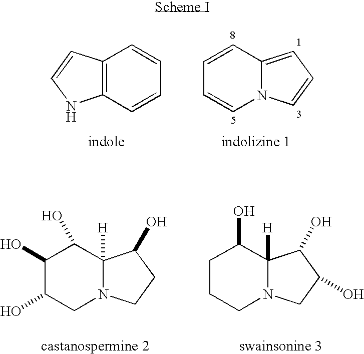

The most interesting of these aza-heterocycles are the fully saturated naturally occurring indolizidines of which castanospermine⁵ 2 and swainsonine⁶ 3 are prototypes. In many cases, these polyhydroxyindolizidine alkaloids have been found to possess a broad range of desirable pharmacological properties such as strong inhibitors of glycosidases. In addition, naturally occurring indolizines have been employed as antibacterial, antiviral, anti-HIV and anti-inflammatory agents.⁴⁻⁶

Synthetic indolizines have been reported to play important roles as pharmaceutical agents as well as synthetic dyes for photosensitive recording materials.⁷ Notably, aminoalkyloxybenzenesulfonylindolizine compounds (e.g Fantofarone, SR33557) have been used for the treatment of angina pectoris, hypertension and arrhythmia.⁸ Additionally, several O-containing indolizines have been screened and identified as possessing strong anti-oxidant effects that prevent the initiation of processes that lead to DNA damage.⁹ 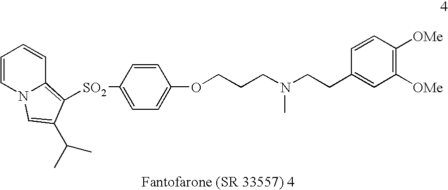
Previous Methods of Synthesis:

Due to the inherent biological activity and therapeutic potential of the substituted indolizine derivatives in the treatment of human diseases, various new methods for their synthesis have been developed. The Scholtz and Tschitschibabin condensation reactions of 2-alkyl-substituted pyridines with acid anhydrides and 2-haloketones have long been known and have proven to be quite valuable in indolizine synthesis.^{10,11} In addition the 1,3- and 1,5 dipolar cycloaddition reactions of pyridinium ylides with various olefinic and acetylenic dipolarphiles continue to be one of the fundamental ways to construct substituted indolizine derivatives. ^{3,10-11}

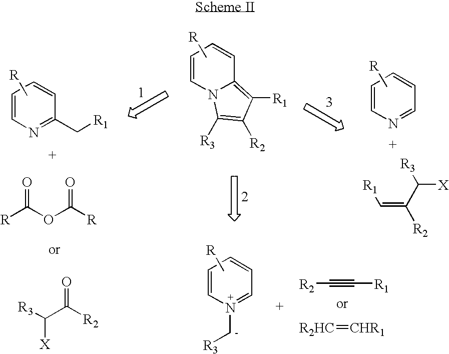

Despite their conciseness, the above-mentioned methods have major drawbacks that diminish their attractiveness. The Tschitschibabin appoach has not been successfully utilized in the synthesis of indolizines that do not possess substituents on the pyrrole ring.¹¹ The cycloaddition method lacks flexibility because it requires the olefinic or acetylenic dipolarphiles to bear two relatively small deactivating groups. As such, no cycloaddition products have been isolated with non-activated dipolarphiles. Clearly, such requirement sets limitations on the substitution patterns at the 1,2 and 3 positions of indolizine nucleus. Therefore an alternate method for the preparation of indolizines that allows functional groups variation on indolizine nucleus is highly desirable.

Accordingly, there is a need for new ways of synthesizing indolizines and benzoindolizine compounds.

### SUMMARY OF THE INVENTION

To our knowledge, no synthesis of mono-substituted 3-alkoxymethyl indolizines and 1-alkoxy-pyrrolo[1,2-a]quinolines has appeared in the chemical literature. We herein describe a new and general synthesis of 3-alkoxymethyl indolizine and the closely related (and in parts, identical) 1-alkoxymethyl-pyrrolo[1,2-a]quinoline. In preferred embodiments, this one-pot synthetic sequence makes use of less expensive, easily available starting materials and milder reaction conditions that offer to reduce the time, waste, and cost associated with synthesis of the above-mentioned N-bridgehead heterocycles.

Thus a first aspect of the present invention is a method of making a compound of Formula I: 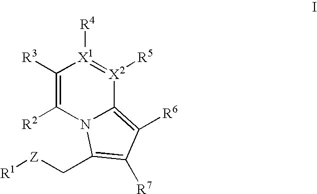
wherein:

X¹ and X² are each independently N or C, subject to the provisos that R⁴ is absent when X¹ is N and R⁵ is absent when X² is N;

Z is O or S;

R¹ is selected from the group consisting of H, alkyl, alkenyl, arylalkyl, alkoxyalkyl alkylthioalky, aryloxyalkyl, alkenyloxyalkyl, silyl, siloxyalkyl, tetrahydropyranyl, tetrahydrothiopyranyl, 1,4-Dioxan-2-yl, tetrahydrofuranyl, tetrahydrothiofumyl, benzyl, p-(methylsulfinyl) benzyl, 2-picoyl, 4-picoyl, 2-quinolinylmethyl, 1-pyrenylmethyl-, 9-(9-phenyl)xanthenyl-, naphthanyl-, cyclodextrin-, and boron compounds (particularly carboranes, including o, m and p-carboranes), halo, and solid supports (or any of the substituents given in connection with R² through R⁷ below);

R², R³, R⁴, R⁵, R⁶ and R⁷ are each independently selected from the group consisting of H, akyl, halo, alkenyl, alkoxy, alkoxyalkyl, alkoxycarbonyl, alkoxycarbonylalkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, haloalkyl, hydroxy, hydroxyalkyl, mercapto, and nitro;

or R² and R³ together form a group of the formula: 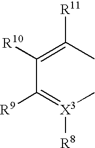
wherein:

X³ is N or C, subject to the proviso that R⁸ is absent when X³ is N;

R⁸, R⁹, R¹⁰, and R¹¹ are each independently selected from the group consisting of H, halo, alkyl, alkenyl, alkoxy, alkoxyalkyl, alkoxycarbonyl, alkoxycarbonylalkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, haloalkyl, hydroxy, hydroxyalkyl, mercapto, and nitro;

said method comprising reacting a compound of Formula II 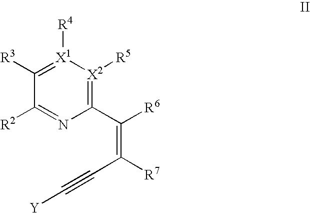
wherein Y is H, alkyl, alkenyl, aryl, or trialkylsilyl and R², R³, R⁴, R⁵, R⁶ and R⁷ are as given above,

with a compound selected from the group consisting of R¹OH and R¹SH, where R¹ is as given above, in the presence of a base to produce said compound of Formula I.

Another aspect of the invention is a method of making a compound of Formula II by reacting a compound of Formula III: 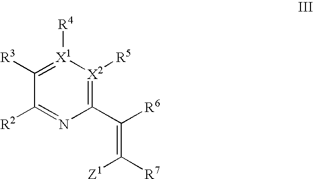
where Z¹ is halo and R², R³, R⁴, R⁵, R⁶ and R⁷ are as given above,

with (trialkylsilyl)acetylene in the presence of a base and a transition metal complex to produce a compound of Formula II.

In some embodiments the (trialkylsilyl)acetylene is (trimethylsilyl)acetylene; in some embodiments the base is triethylamine; in some embodiments the transition metal complex is a palladium complex.

A further aspect of the invention is a compound of Formula Ia: 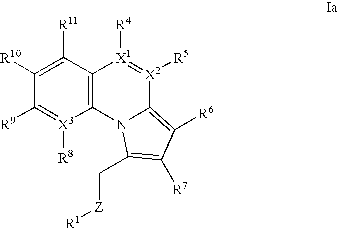
wherein:

X¹, X² and X³ are each independently N or C, subject to the provisos that R⁴ is absent when X¹ is N; R⁵ is absent when X² is N, and R⁸ is absent when X³ is N;

Z is O or S;

R¹ is as described herein; and

R², R³, R⁴, R⁵, R⁶, R⁷, R⁸, R⁹, R¹⁰, and R¹¹ are as described herein.

A further aspect of the invention is a compound of the formula IIa: 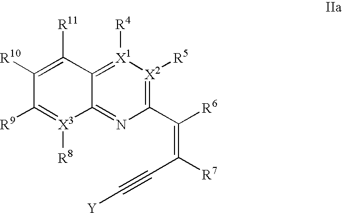
wherein:

X¹, X² and X³ are each independently N or C, subject to the provisos that R⁴ is absent when X¹ is N; R⁵ is absent when X² is N, and R⁸ is absent when X³ is N;

Y is H, alkyl, alkenyl, aryl, or trialkylsilyl; and

R², R³, R⁴, R⁵, R⁶, R⁷, R⁸, R⁹, R¹⁰, and R¹¹ as described above.

The foregoing and other objects and aspects of the present invention are explained in greater detail in the specification set forth below.

### DETAILED DESCRIPTION OF THE PREFERRED EMBODIMENTS

1. Definitions. As used throughout this specification and the appended claims, the following terms have the following meanings:

“Alkyl,” as used herein, refers to a straight or branched chain hydrocarbon, or cyclic hydrocarbon, containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. The alkyl groups of the present invention may be optionally substituted with 0, 1 or 2 substituents that are members selected from the group consisting of alkoxy, alkoxycarbonyl, alkylcarbonyl, alkylcarbonyloxy, alkylsulfonyl, alkylthio, carboxy, carboxyalkyl, cyano, cyanoalkyl, formyl, halogen, hydroxy, alkoxycarbonylNR_{g}, alkylNR_{g} wherein R_{g} is a member selected from the group consisting of hydrogen and alkyl.

“Halo” or “halogen,” as used herein, refers to —Cl, —Br, —I or —F.

“Alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

“Alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

“Alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

“Alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

“Alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

“Alkylsulfonyl,” as used herein, refers to an alkyl group, as defined herein, appended to the parent molecular moiety through a sulfonyl group, as defined herein. Representative examples of alkylsulfonyl include, but are not limited to, methylsulfonyl and ethylsulfonyl.

“Alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl. The alkynyl groups of this invention can be substituted with 0, 1, 2, or 3 substituents independently selected from alkoxy, alkoxyalkyl, alkoxycarbonyl, alkoxycarbonylalkyl, alkylcarbonyl, alkylcarbonylalkyl, heterocycle, heterocyclealkyl, hydroxy, and hydroxyalkyl.

“Allenyl,” as used herein, refers to a straight or branched chain hydrocarbon containing from 3 to 10 carbons and containing two double bonds between three contiguous carbons formed by the removal of four hydrogens. Representative examples of alkenyl include, but are not limited to, propa-1,2 dienyl, penta-1,2 dienyl, penta-2,3 dienyl, hexa-1,2-dienyl and the like.

“Aryl,” as used herein, refers to a monocyclic-ring system, or a bicyclic- or a tricyclic-fused ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl. The aryl groups of the present invention can be substituted with 0, 1, 2, or 3 substituents independently selected from alkyl, alkenyl, alkoxy, alkoxyalkyl, alkoxycarbonyl, alkoxycarbonylalkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, halogen, haloalkyl, heterocycle, heterocyclealkyl, hydroxy, hydroxyalkyl, mercapto, nitro, or phenyl, R_{E}R_{F}N—, R_{G}R_{H}NC(O)—, and R_{G}R_{H}NS(O)₂—, wherein R_{E} and R_{F} are each independently selected from the group consisting of alkyl, alkylcarbonyl, alkoxycarbonyl, alkylsulfonyl, and R_{G} and R_{H} are each independently selected from the group consisting of hydrogen and alkyl.

“Arylalkoxy,” as used herein, refers to an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

“Arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

“Arylcarbonyl,” as used herein, refers to an aryl group, as defined herein, appended to the parent molecular moiety through a carbonyl group, as defined herein. Representative examples of arylcarbonyl include, but are not limited to, benzoyl and naphthoyl.

“Carbonyl,” as used herein, refers to a —C(O)— group.

“Carboxy,” as used herein, refers to a —CO₂H group.

“Cyano,” as used herein, refers to a —CN group.

“Cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

“Cycloalkyl,” as used herein, refers to a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo(3.1.1)heptane, bicyclo(2.2.1)heptane, bicyclo(2.2.2)octane, bicyclo(3.2.2)nonane, bicyclo(3.3.1)nonane, and bicyclo(4.2.1)nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo(3.3.1.0³,7)nonane and tricyclo(3.3.1.1³,7)decane (adamantane). The cycloalkyl groups of this invention may be substituted with 0, 1, 2 or 3 substituents selected from alkyl, alkylcarbonyl, alkoxy, alkoxycarbonyl, alkenyl, alkynyl, aryl, cyano, halogen, hydroxy, hydroxyalkyl, nitro, R_{E}R_{F}N—, R_{G}R_{H}NC(O)—, and R_{G}R_{H}NS(O)₂—, wherein R_{E} and R_{F} are each independently selected from the group consisting of alkyl, alkylcarbonyl, alkoxycarbonyl, alkylsulfonyl, and R_{G} and R_{H} are each independently selected from the group consisting of hydrogen and alkyl.

“Cycloalkylalkyl,” as used herein, refers to a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

“Haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

“Haloalkenyl,” as used herein, refers to at least one halogen, as defined herein, appended to the parent molecular moiety through an alkenyl group, as defined herein. Representative examples of haloalkenyl include, but are not limited to, chloroethylenyl, 2-fluoroethylene, trifluorobutenyl, and dichloropropenyl.

“Heterocycle” or “heterocyclic,” as used herein, refers to a monocyclic, bicyclic, or tricyclic ring system. Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered ring have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic ring systems fused to an aryl group as defined herein, a cycloalkyl group as defined herein, or another monocyclic ring system. Representative examples of bicyclic ring systems include but are not limited to, for example, benzimidazolyl, benzodioxinyl, benzothiazolyl, benzothienyl, benzotriazolyl, benzoxazolyl, benzofuranyl, benzopyranyl, benzothiopyranyl, cinnolinyl, indazolyl, indolyl, 2,3-dihydroindolyl, indolizinyl, naphthyridinyl, isobenzofuranyl, isobenzothienyl, isoindolyl, isoquinolinyl, phthalazinyl, 4H-pyrido(1,2-a)pyrimidin-4-one, pyranopyridinyl, quinolinyl, quinolizinyl, quinoxalinyl, quinazolinyl, -tetrahydroisoquinolinyl, tetrahydroquinolinyl, and thiopyranopyridinyl. Tricyclic rings systems are exemplified by any of the above bicyclic ring systems fused to an aryl group as defined herein, a cycloalkyl group as defined herein, or a monocyclic ring system. Representative examples of tricyclic ring systems include, but are not limited to, acridinyl, carbazolyl, carbolinyl, dibenzo(b,d)furanyl, dibenzo(b,d)thienyl, naphtho(2,3-b)furan, naphtho(2,3-b)thienyl, phenazinyl, phenothiazinyl, phenoxazinyl, thianthrenyl, thioxanthenyl and xanthenyl.

Heterocycles can be substituted with 0, 1, 2 or 3 substituents independently selected from alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, halogen, haloalkyl, hydroxy, hydroxyalkyl, mercapto, nitro, phenyl, R_{E}R_{F}N—, R_{G}R_{H}NC(O)—, and R_{G}R_{H}NS(O)₂—, wherein R_{E} and R_{F} are each independently selected from the group consisting of alkyl, alkylcarbonyl, alkoxycarbonyl, alkylsulfonyl, and R_{G} and R_{H} are each independently selected from the group consisting of hydrogen and alkyl.

“Heterocyclealkyl,” as used herein, refers to a heterocycle, as defined herein, appended to the parent molecular moiety through an alkyl group, as defined herein. Representative examples of heterocyclealkyl include, but are not limited to, pyridin-3-ylmethyl and 2-pyrimidin-2-ylpropyl and the like.

“Hydroxy,” as used herein, refers to an —OH group.

“Hydroxyalkyl,” as used herein, refers to a hydroxy group, as defined herein, appended to the parent molecular moiety through an alkyl group, as defined herein. Representative examples of hydroxyalkyl include, but are not limited to, 2-hydroxyethyl, 2-hydroxypropyl, 3-hydroxybutyl and the like.

“Heterocyclecarbonyl,” as used herein, refers to a heterocycle, as defined herein, appended to the parent molecular moiety through an carbonyl group, as defined herein. Representative examples of heterocyclecarbonyl include, but are not limited to, pyridin-3-ylcarbonyl and 2-pyrimidin-2-ylcarbonyl and the like.

“Solid support” as used herein may be any suitable polymeric or nonpolymeric, organic or inorganic, solid support, in any suitable form such as a particle, bead, or gel. Exemplary materials include but are not limited to solid phase synthesis resins, silica, glass, polymer matrixes such as agarose gels, carbohydrates and aza-sugars, etc.

“Nitro,” as used herein, refers to a —NO₂ group.

The disclosures of all United States patents cited herein are to be incorporated by reference herein in their entirety.

2. Compounds of Formula I.

In general, compounds of Formula I are produced by reacting a compound of Formula II with a compound such as R¹OH, R¹SH. The reaction is typically carried out in the presence of a base such as KOH, K₂CO₃, or KF or CsF, wth KF or CsF currently preferred. The reaction may be carried out at any suitable time or temperature, though elevated temperatures result in shorter reaction times and, in some cases, higher yields. Thus temperatures of at least 25° C., 30° C. or 40° C., up to 80° C., 100° C., or even 150° C. or more are preferred. The reaction may be conveniently carried out under reflux conditions. Reaction times may range from one half hour to 24 hours or more. The reaction may be carried out under atmospheric conditions in any suitable solvent or solvent mixture, preferably non-chlorinated solvents such as toluene, benzene, acetonitrile and tetrahydrofuran (THF) and DMF. Toluene is the preferred solvent when other reagent (e.g. thiols) are used.

Particular examples of compounds of Formula I are as follows: 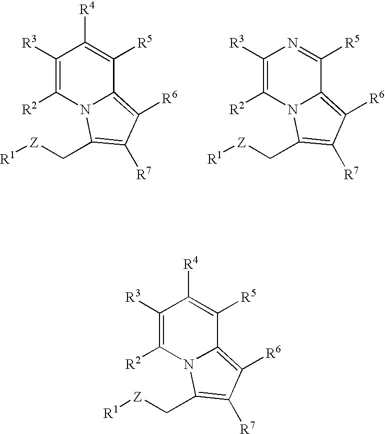
where Z and R¹ through R⁷ are as given herein.

R¹ in formula I particularly includes: unsubstituted alkyl, substituted alkyls such as alkoxyalkyls (e.g. CH₃OCH₂O—) (MOM) methoxymethy and methylthiomethyl (MTM) MeSCH₂O—, benzyloxymethyl (BOM)-; Guaiacolmethyl (GUM) (2-MeO—C₄H₆—OCH₂O—); 4-pentenyloxymethy (POM) CH₂═CH₂CH₂CH₂CH₂OCH₂—, unsubstituted and substituted silyl (TMS, TES, TBDMS, TIPS, TBDPS), siloxyalkyl (e.g., RR¹SiO(CH₂)ₙ where n is, for example, from 1-10, tetrahydropyranyl, tetrahydrothiopyranyl, 1,4-Dioxan-2-yl, Tetrahydrofuranyl, tetrahydrothiofumyl, benzyl, p-(methylsulfinyl)benzyl, 2-picoyl, 4-picoyl, 2-quinolinylmethyl, 1-pyrenylmethyl-, 9-(9-phenyl)xanthenyl-, naphthanyl-, cyclodextrin-bound, resin-bound (e.g., Wang)—solid phase synthesis, and boron compounds (e.g carboranyl)-.

Additional examples of compounds of Formula I (particularly compounds of Formula Ia) are as follows: 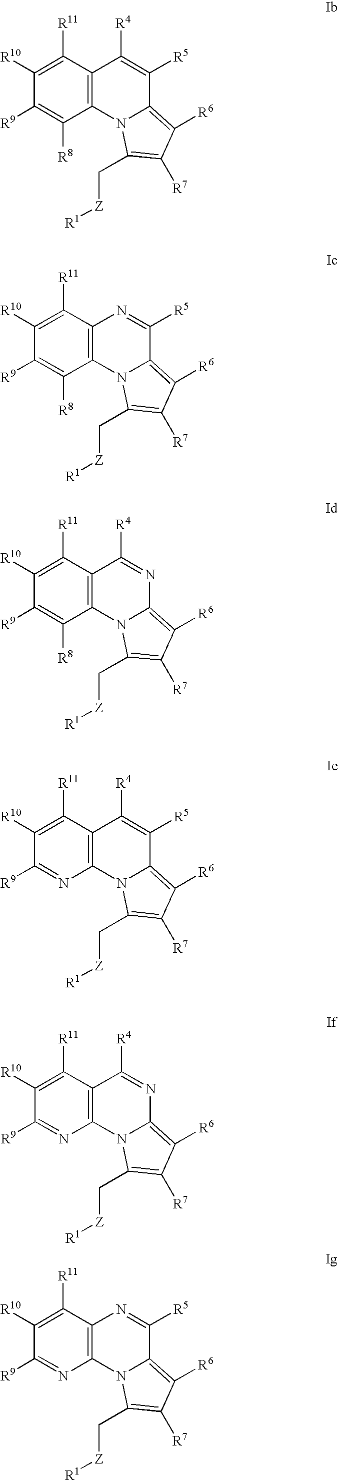
wherein Z and R¹ through R¹¹ are as given herein.

3. Compounds of Formula II.

The reaction by which compounds of Formula II are formed is, in general, carried out under Wittig followed by Sonogashira reaction conditions. Wittig and Sonogashira reaction conditions are known. See, e.g., Ref. 13; see also U.S. Pat. Nos. 6,667,287 and 6,458,985. In general, the reaction is carried out in the presence of base and a transition metal catalyst. Bases suitable for the reaction may be, for example, an organic base such as a primary, secondary or tertiary amine. Non-limiting examples include triethylamine, diisopropylamine, 1,8-diazabicyclo-[5.4.0-]-undec-7-ene (DBU), 1,5-diazabicyclo-[4,3,0]-non-5-ene (DBN), or 1,4-diazabicyclo-[2.2.2]-octane (DABCO). Alternatively, an inorganic base may be used, such as an alkali metal or alkaline earth metal salt, such as a carbonate, bicarbonate or acetate salt. Metal catalysts may be in the form of a salt or a complex with organic ligands. Particularly suitable metal catalysts are, for example, the Group VIII metals, preferably Pd(0) complexes or a Pd(II) salt. The ligands may be selected from, for example, phosphorus-containing ligands, such as triphenylphosphine (PPh₃) and 1,2-bis(diphenyl-phosphino)eth-ane (dppe). Preferred palladium catalysts include Pd(PPh₃)₂Cl₂, Pd(PPh₃)₄ and Pd(OAc)₂. The reaction is performed in the presence of a Cu(I) salt, such as a Cu(I) halide, Cu₂O, and CuCN, preferably CuI or CuCl. Suitable organic solvents include, but are not limited to, dioxane, tetrahydrofuran (THF) dimethylformamide (DMF), acetonitrile, dimethylsulfoxide, and other polar aprotic solvents or mixtures thereof. For further discusion of the Sonogashira reaction, see Sonogashira, K. et al., Tetrahedron Lett. 1975, 4467-4470; Sonogashira, K. In Comprehesive Organic Synthesis, Trost, B. M.; Fleming, L., Eds., Pergamon Press: New York, 1991, Vol. 3, chapter 2.4; Liao, Y. et al., Tetrahedron Lett. 2001, 42, 1815-1818; Nicolaou, K. et al., Acc. Chem. Res. 1992, 25, 497-503; Takeuchi, R. et al., J. Org. Chem. 2000, 65, 1558-1561; Arterbum, J. B. et al., Tetrahedron Lett. 2000, 41, 839-842; Gan, Z.; et al., Tetrahedron Lett. 2000, 41, 1155-1159; Godt, A. et al., Org. Chem. 2000, 65,2837-2842; Yoshimura, F. et al., Tetrahedron Lett. 1999, 40, 8281-8286; Tretyakov, E. et al., J. Chem. Soc., Perkin Trans. 1, 1999, 3713-3720; Thorand, S. et al., J. Org. Chem. 1998, 63, 8551-8553; and Sonogashira, K. in Metal-Catalyzed Cross-Coupling Reactions; Diederich, F., Stang, P. J., Wiley-VCH: New York, 1998. See generally US Patent Application 2004/0110949.

Alkyl can be generated as as R¹ by a number of methods. The acetylenic hydrogen (R1═H) can be readily substituted by alkyl halides (e.g., R—X where X═Br, I) in the presence of a suitable base or other organic halides such as alkenyl, aryl, acyl and aminocarbonyls in the presence of copper (I) iodide/bis(triphenylphosphine) dichloride in amines.

Particular examples of compounds of Formula II are as follows: 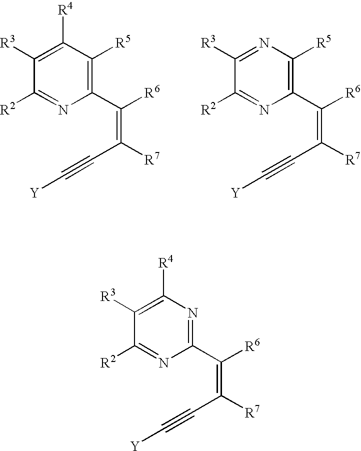
where substituents Y and R² through R⁷ are as given herein.

Still additional examples of compounds of Formula II (particularly compounds of Formula IIa) include the following: 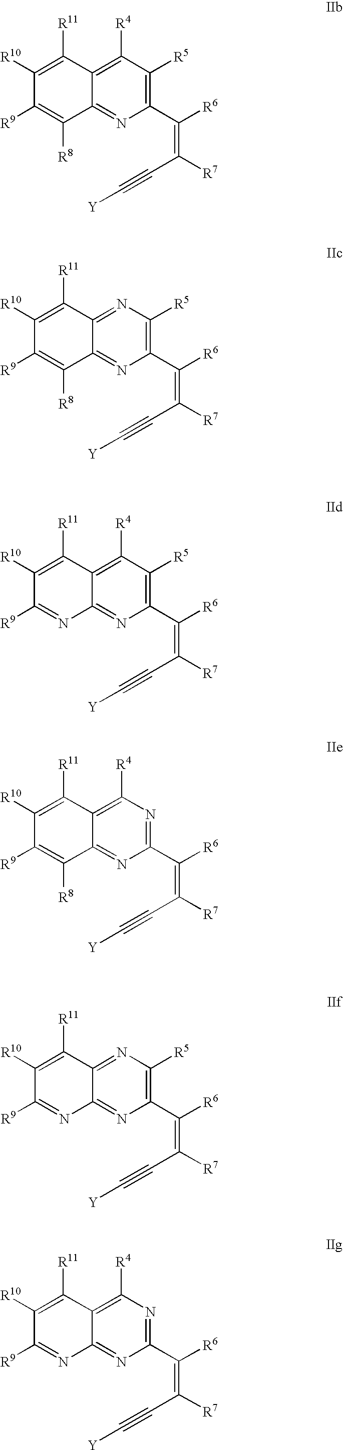
where substituents Y and R² through R¹¹ are as given herein.

3. Utility. Compounds of Formula I are useful as dyes (e.g., for photosensitive recording materials), as spectral sensitizers, as inhibitors of glycosidases, and as antibacterial, antiviral, and anti-inflammatory agents. Compounds of Formula I are useful as intermediates for the manufacture of compunds that have pharmacological activity in the treatment of human or animal subjects (e.g., central nervous system depressants, calcium entry blockers, cardiovascular agents such as for the treatment of angina pectoris, hypertension and arrhythmia, spectral sensitizers). Compounds of Formula II are useful as intermediates for the manufacture of compounds of Formula I.

4. Hydrogenation of compounds of Formula I. Compounds of Formula I provide a convenient route to the hydrogenated counterparts thereof. Thus, a further aspect of the invention is a method of making a compound of Formula XI: 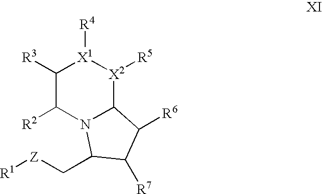
wherein:

X¹ and X² are each independently N or C, subject to the provisos that R⁴ is absent when X¹ is N and R⁵ is absent when X² is N;

Z is O or S;

R¹ is selected from the group consisting of H, alkyl, alkenyl, arylalkyl, alkoxyalkyl alkylthioalkylaryloxyalkyl; alkenyloxyalkyl; silyl, siloxyalkyl, tetrahydropyranyl, tetrahydrothiopyranyl, 1,4-Dioxan-2-yl, tetrahydrofuranyl, tetrahydrothiofurnyl, benzyl, p-(methylsulfinyl) benzyl, 2-picoyl, 4-picoyl, 2-quinolinylmethyl, 1-pyrenylmethyl-, 9-(9-phenyl)xanthenyl-, naphthanyl-, cyclodextrin-, boron compounds, halo, and solid supports;

R², R³, R⁴, R⁵, R⁶ and R⁷ are each independently selected from the group consisting of H, akyl, halo, alkenyl, alkoxy, alkoxyalkyl, alkoxycarbonyl, alkoxycarbonylalkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, haloalkyl, hydroxy, hydroxyalkyl, mercapto, and nitro;

or R² and R³ together form a group of the formula: 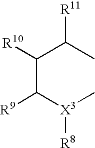
wherein:

X³ is N or C, subject to the proviso that R⁸ is absent when X³ is N;

R⁸, R⁹, R¹⁰, and R¹¹ are each independently selected from the group consisting of H, halo, alkyl, alkenyl, alkoxy, alkoxyalkyl, alkoxycarbonyl, alkoxycarbonylalkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, haloalkyl, hydroxy, hydroxyalkyl, mercapto, and nitro;

said method comprising hydrogenating a compound of Formula I as described above and herein to produce said compound of Formula XI. While Formula XI shows the compounds produced by hydrogenation in fully hydrogenated form, it will be appreciated that the hydrogenation may be partial, that, for example, and 1, 2, 3 or 4 of the double bonds found in the compounds of Formula I may be retained as long as at least one double bond is hydrogenated.

Hydrogenation may be carried out in accordance with known techniques by use of an appropriate catalyst, reducing agents, temperatures and pressures.

The hydrogenated and partially hydrogenated compounds of Formula XI are useful for like purposes as the compounds of Formula I.

The present invention is explained in greater detail in the non-limiting examples set forth below.

### EXPERIMENTAL

### Synthesis of the Starting Material

A simple yet high yielding synthetic sequence has been devised which provides access to geometrically pure (Z)-pyridne-containing vinylacetylenes as starting materials (Scheme III).¹²⁻¹⁴ Thus, Wittig olefination of commercially available heteroaromatic aldehydes 5 with bromomethyl triphenylphosphonium bromide yielded chiefly Z-heteroaryl-vinylbromides 6. Cross coupling of the Z-monobromides with (trimethylsilyl) acetylene under Sonogashira conditions¹³ yielded the trimethylsilyl-capped enynes 7. The treatment of protected enynes 7 with basic alcohol solutions or ionic fluorides at 0° C. afforded the desired 2-pyridine containing vinylacetylenes 8 exclusively. It should be noted that the temperature should preferably be kept low (≦0° C.) throughout the reaction, otherwise in the presence of basic alcoholic solution or ionic fluorides the cyclization of 8 to 10 (Scheme IV) is observed and the yield of enyne 8 is greatly lowered.

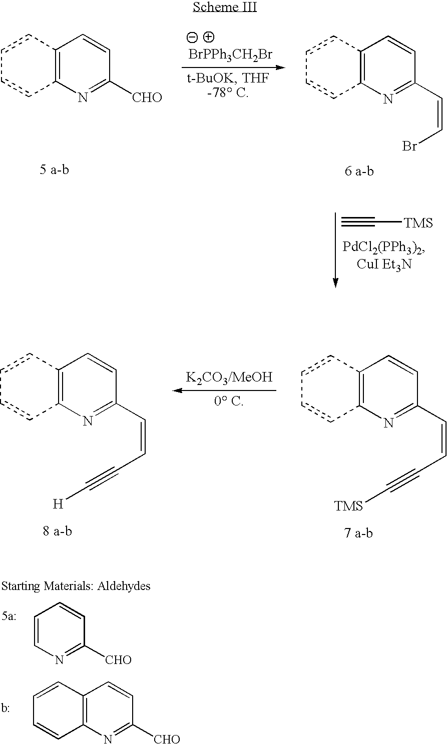

New Method for Synthesis of 3-substututed Indolizines. In the course of desilylation of Z-2-[4-(Trimethyl-silanyl)-but-1-en-3-ynyl]-pyridine 7a to 2-pyridine vinylacetylene 8a at room temperature using the standard K₂CO₃/MeOH procedure, we observed (TLC analysis) the conversion of the starting material initially to the expected 2-pyridine vinylacetylene 8a which with time completely was consumed to a new product 10 a of lower polarity (Scheme IV). The unanticipated product 10a was unambiguously identified as 3-methoxymethylindolizine. This result was indeed surprising since this process was expected to give only the desilylated pyridine enyne 8a. Since there are no existing general routes to 3-substituted alkoxymethylindoliiznes and 1-substituted alkoxymethylpyrrole[1,2-a]quinolines, we have tactically developed the procedure described herein as a new practical route to a variety of 3-substituted alkoxymethylindolizines and benzoindolizines.

We have observed that simply heating (Z)-2-[4-(Trimethyl-silanyl)-but-1-en-3-ynyl]-pyridine 7a in the presence of various basic alcohol solutions or ionic fluorides smoothly afford the corresponding 3-substituted alkoxymethylindolizine derivatives in good to excellent yields (40-100%).

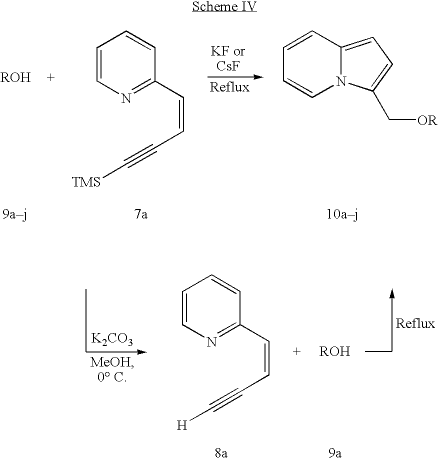

We have investigated several aspects of the reaction conditions and noticed that factors such as temperature and type of alcohol and base used greatly affect the outcome of these reactions. We found that KF or CsF along with heating the reaction mixture to reflux resulted in shorter reaction times and higher yields. The results of this study are summarized in Table 1. In some cases, the desired compounds were simply extracted from the reaction mixture with excellent purity, thereby eliminating the need for further chromatographic purification. The structures of compounds 10a-j were supported by NMR spectroscopy, elemental analysis and/or mass spectrometry (HRMS).

TABLE 1

| | | | | | Yield % | |
|---|---|---|---|---|---|---|
| Entries | Alcohols 9 | Products | 10 | Base | (Method) | Time (hr) |
| 1 | CH₃OH 9a | 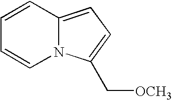 | 10a | KF | 90 (A) | 1 hr |
| 2 | CH₃CH₂OH 9b | 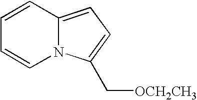 | 10b | KF | 92 (A) | 1 hr |
| 3 | CH₂═CH—CH₂OH 9c | 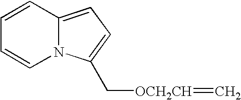 | 10c | KF | 100 (A) | 0.75 hr |
| 4 | (CH₃)₂CHOH 9d | 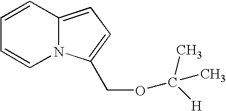 | 10d | KF | 94 (A) | 1.5 hr |
| 5 | 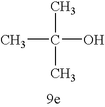 | 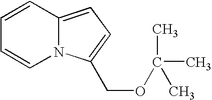 | 10e | KF | 84 (A) | 7 hr |
| 6 | CH₃(CH₂)₃CHOH 9f | 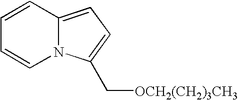 | 10f | CsF | (B) | 1 hr |
| 7 | CH₃(CH₂)₈CH₂OH 9g | 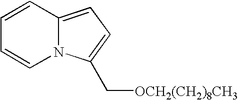 | 10g | CSF | (C) | 24 hr |
| 8 | 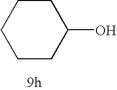 | 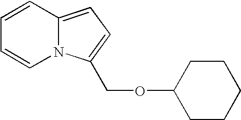 | 10h | CsF | 68 (B) | 1 hr |
| 9 | 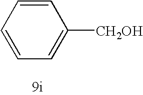 | 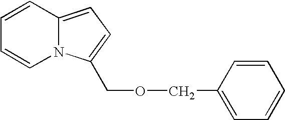 | 10i | CsF | 40 (C) | 2 hr |
| 10 | CH₃OD 9j | 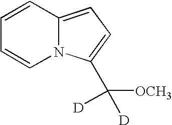 | 10j | KF | (A) | 1 hr |

Preparation of Benzoindolizines. This reaction can also be extended to the preparation of 1-substituted pyrrole[1,2-a]quinolines (benzoindolizines). Thus, when the model substrate 2-quinoline TMS 7b was treated with 2.0 equivalent of KF or 1.5 equivalent of CsF in different alcohols, the conversion to the benzoindolizine 11 derivatives was complete (Table 2). For higher boiling and expensive alcohols such as benzyl alcohol, n-decanol or deuterated and fluorinated alcohols the use of the alcohol as solvent was undesirable. It has been demonstrated that such transformations proceeded smoothly using CsF and ten equivalents of the higher boiling alcohol in refluxing anhydrous toluene (Scheme V).

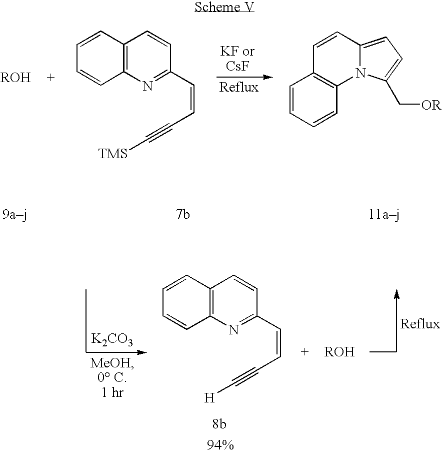

TABLE 2

| | | | | | Yield % | |
|---|---|---|---|---|---|---|
| Entries | Alcohols 9 | Products | 11 | Base | (Method) | Time |
| 1 | CH₃OH 9a | 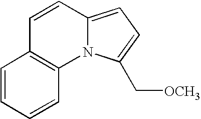 | 11a | KF | 95 (A) | 1 hr |
| 2 | CH₃CH₂OH 9b | 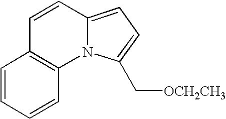 | 11b | KF | 96 (A) | 1 hr |
| 3 | CH₂═CH—CH₂OH 9c | 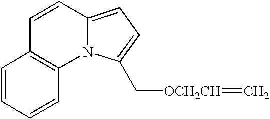 | 11c | KF | 100 (A) | 0.75 hr |
| 4 | (CH₃)₂CHOH 9d | 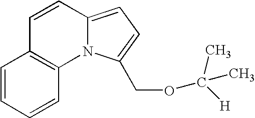 | 11d | KF | 86 (A) | 1.5 hr |
| 5 | 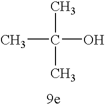 | 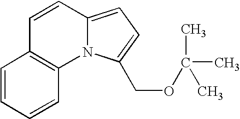 | 11e | KF | 97 (A) | 7 hr |
| 6 | CH₃(CH₂)₃CHOH 9f | 11f | CsF | 57 (B) | 1 hr | |
| 7 | CH₃(CH₂)₈CH₂OH 9g | 11g | CsF | C | 24 hr | |
| 8 | 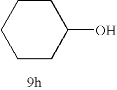 | 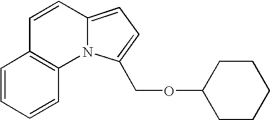 | 11h | CsF | 77 (B) | 1 hr |
| 9 | 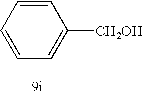 | 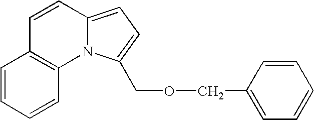 | 11i | CsF | 47 (C) | 2 hr |
| 10 | CH₃OD | 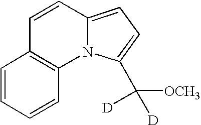 | 11j | KF | 87 (A) | 1 hr |

General Reaction Procedures. All operations involving air-sensitive reagents or organometallics were conducted under a nitrogen atmosphere. All glassware for reactions were dried in an oven overnight at 135° C., assembled hot, and cooled under a stream of nitrogen. Temperatures of 0° C. were obtained with an ice/water bath; temperatures of −78° C. were obtained with acetone/dry ice bath. Sodium chloride solution and sodium bicarbonate solution refer to the saturated solutions. Drying and concentration refers to the drying of an ethereal solution over sodium sulfate or magnesium sulfate, filtration, and rotary evaporation of volatile solvents under reduced pressure (water aspirator at 40 mmHg). Alcohols 9a-j are commercially available and in some occasions were distilled before use. Melting points were obtained with Mel temp capillary melting point apparatus and are uncorrected.

Spectra Data. Proton nuclear magnetic resonance (¹H NMR) spectra were recorded on Varian Unity 300 or Varian Unity 500 MHz spectrometers. Samples were dissolved in deuteriochloroform, 99.8% deuterium (Aldrich Chemical Co.). Tetramethylsilane (TMS) or residual chloroform were used as internal standards at 0.0 or 7.24 ppm, respectively. Chemical shifts are reported in δ values, and coupling constants (J) are reported in hertz. Carbon NMR (¹³C NMR) spectra were recorded on a Varian Unity 300 and Unity 500 spectrometers in deuteriochloroform and are broadband unless otherwise stated. Chemical shifts are reported in δ units downfield from TMS with deuteriochloroform as internal reference at 77.0 ppm.

Chromatographic Separations. Flash chromatography was performed according to the procedure of Still et al. (Still, W. C.; Kahn, M.; Mitra, A. J. Org. Chem. 1978, 43, 2923. (b) Brennan, M. R.; Erickson, K. L. J. Org. Chem. 1982, 47, 3917).

Thin-layer chromatography (TLC). TLC was accomplished with precoated 60F 254 plastic plates (Aldrich brand) and visualized by UV light at 254 nm, development in an iodine chamber or by phosphomolybdic acid 1% spray in ethanol and heating.

### Synthesis of 3-alkoxymethylindolizine and 1-alkoxymethyl-pyrrolo[1,2-a]quinoline 10 and 11

### Example 1

### General Procedure A; Using Alcohols with Low Boiling Point 9a-e, j

2.0 mmol of potassium fluoride (KF) was added to silylated enyne 7a or 7b (1 mmol) in the appropriate alcohol (20 mL). The mixture was heated and kept under reflux until 7a or 7b was no longer detectable (TLC analysis). The solvent (low boiling alcohol) was removed in vacuo. The residue was dissolved in petroleum ether or hexanes and washed with water (3×30 mL). The organic layer was dried over sodium sulfate or magnesium sulfate under nitrogen and concentrated to yield the pure products.

### Example 2

### General Procedure B: Using Alcohols with High Boiling Point 9f-9i

The synthetic procedure is similar to General Procedure A. However, the work-up procedure differs. In this case, the reaction mixture was poured into a solution of MeOH/H₂O (1:1) 40 mL and extracted with petroleum ether (3×20 mL). The organic layer was dried over sodium sulfate or magnesium sulfate under nitrogen and concentrated. The crude product was flashed chromatographed on silica gel, silica gel with 3% triethylamine) or alumina (basic) using hexanes/ethyl acetate as eluent.

### Example 3

### General Procedure C: Expensive and/or High Boiling Alcohols

A suspension of silylated enyne 7a or 7b (1 mmol), CsF (1.5 mol) and 10 mmols of appropriate alcohol in anhydrous toluene (20 mL) was well-stirred at reflux until the disapperance of the starting material 7a or 7b. The solvent was evaporated in vacuo, and the residue purified by flask chromatography (SiO₂, SiO₂ with 5% triethylamine) or alumina (basic) using petroleum ether/ethyl acetate as eluent.

### Example 4

### Synthesis of (Z)-2-(β-bromovinyl) pyridine 6a

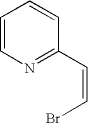

To a cooled (−78° C.) suspension of bromomethyl triphenylphosphonium bromide (25.27 g, 0.050 mol) in dry THF (150 ml) under a nitrogen atmosphere, was added potassium tert-butoxide (6.57 g, 0.050 mol). The resulting yellow mixture was stirred at this temperature for 1 hr. A solution of 2-pyridine carboxyaldehyde (5 mL, 0.042 mol) in dry THF (10 mL) was then introduced via a syringe. The temperature was maintained at −78° C., and the mixture was stirred an additional 5 hrs. The mixture was diluted with 80 mL of petroleum ether, and filtered with vacuum. Evaporation of the solvent and purification by flash column chromatography (silica gel, 30% ethyl acetate in petroleum ether) gave vinyl bromide (7.29 g, 95%) as a yellow oil. The product contains Z and E isomers in 9:1 Z/E. ¹H NMR (cis) (500 MHz, CDCl₃) δ6.66 (1H, d, J=8.0 Hz), 7.23 (1H, ddd, J=7.5, 5.0, 1.0 Hz), 7.26 (1H, d, J=8.5 Hz), 7.69 (1H, td, J=7.5, 1.5 Hz), 8.01 (1H, dt, J=8.0, 1.0 Hz), 8.64 (1H, dt, J=5.0, 1.0 Hz); ¹³C NMR (75 MHz, CDCl₃) δ 109.4, 122.9, 123.9, 133.4, 136.1, 149.7, 154.0.

### Example 5

### Synthesis of (Z)-2-(4-Trimethylsilanyl-but-1-en-3-ynyl)-pyridine 7a

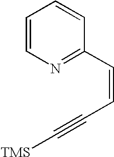 To a solution of monobromide 6a (2.60 g, 14.1 mmol) in triethylamine (50 mL) was added bistriphenylphosphine palladium chloride (0.19 g, 0.28 mmol). After stirring for 10 min, copper iodide (0.13 g, 0.71 mmol) and trimethylsilylacetylene (2.40 mL, 16.9 mmol) were added to the mixture. The resulting mixture was stirred further for 6 hrs at room temperature. After evaporation of the solvent at reduced pressure the residue was dissolved in diethyl ether (40 mL) and filtered through Celite. The ether solution was washed with concentrated ammonium hydroxide (15 mL), water, dried over sodium sulfate, and the solvent was removed under reduced pressure. The residue was purified by flash column chromatography on silica gel using petroleum ether/ethyl acetate (9:1) as eluent to afford the titled compound. Yield 2.83 g (100%) as a yellow oil. ¹H NMR (300 MHz, CDCl₃) δ 0.25 (9H, s), δ 5.96 (1H, d, J=12.3 Hz), 6.88 (1H, d, J=12.3 Hz), 7.24 (1H, ddd, J=6.6, 4.8, 1.2 Hz), 7.69 (1H, td, J=6.4, 2.0 Hz), 8.46 (1H, d, J=8.1 Hz), 8.60 (1H, d, J=4.8 Hz); ¹³C NMR (125 MHz, CDCl₃) δ0.0, 103.3, 104.2, 111, 123.2, 136, 140.9, 149.6, 155.3; MS (EI) m/z (rel. intensity) 201 (M⁺, 0), 200 (base), 186 (60), 170 (17), 156 (20), 141 (10), 132 (35), 130 (6), 106 (6), 83 (5), 78 (5), 67 (5), 53 (5). HRMS calculated for C₁₂H₁₅NSi 201.0974, found 201.0935; IR: (neat cm⁻¹) 3057, 2966, 2341, 2141, 2067, 1584, 1392, 1250, 1153, 1050, 1020, 986, 836

### Example 6

### Synthesis of (Z)-2-(β-bromovinyl) quinoline 6b

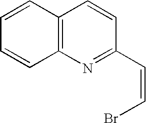

Compound 27 was synthesized analogously to 6a, from bromomethyl triphenylphosphonim bromide (20.81 g, 47.72 mmol), potassium tert-butoxide (5.36 g, 47.72 mmol) and 2-quinoline carboxaldehyde (6.00 g, 38.18 mmol) in 200 mL of THF. Purification by flash column chromatography on silica gel (10:1 petroleum ether/ethyl acetate) gave 6.59 g (80%) product as yellow oil. The product contains cis and trans isomers in 13:1 cis/trans. ¹H NMR (300 MHz, CDCl₃) δ6.79 (1H, d, J=8.1 Hz), 7.45 (1H, d, J=8.1 Hz), 7.55 (1H, td, J=6.9, 1.2 Hz), 7.72 (1H, td, J=6.9, 1.5 Hz), 7.81 (1H, dd, J=8.1, 1.2 Hz), 8.07, (1H, d, J=8.4 Hz), 8.11, (1H, d, J=8.7 Hz), 8.17, (1H, d, J=8.7 Hz), ¹³C NMR (75.42 MHz, CDCl₃) δ 110.70, 121.50, 127.14, 127.54, 127.78, 129.61, 130.01, 134.05, 136.14, 148.23, 154.59

### Example 7

### Synthesis of (Z)-2-(4-Trimethylsilanyl-but-1-en-3-ynyl)-quinoline 7b

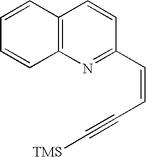

Copper iodide (0.24 g, 1.27 mmol) and bistriphenylphosphine palladium chloride (0.44 g, 0.063 mmol) were added to a solution of vinyl bromide 6b (5.92 g, 25.30 mmol) in triethylamine (100 mL). After this mixture was stirred for 10 min, trimethylsilylacetylene (4.30 mL, 30.36 mmol) was added and the mixture was stirred over night at room temperature. After evaporation of the solvent under reduced pressure, the residue was dissolved in hexanes and filtered through Celite. The hexanes solution was washed with concentrated ammonium hydroxide (15 mL), water, dried over sodium sulfate, and the solvent was removed under reduced pressure. The residue was purified by flash column chromatography on silica gel using 15:1 hexanes/ethyl acetate as eluent. The protected quinoline-enyne was obtained as a yellow solid (4.83 g, 76%). ¹H NMR (300 MHz, CDCl₃) δ0.25, (9H, s), 6.10 (1H, d, J=12.3 Hz), 7.06 (1H, d, J=12.3 Hz), 7.52 (1H, td, J=6.9, 1.2 Hz), 7.70 (1H, td, J=6.9, 1.2 Hz), 7.78 (1H, d, J=8.1), 8.05 (1H, d, J=8.7), 8.13 (1H, d, J=8.7), 8.58 (1H, d, J=8.7); ¹³C NMR (75 MHz, CDCl₃) δ 0.00, 103.16, 104.51, 112.59, 120.93, 127.04, 127.66, 127.75, 129.73, 129.87, 135.96, 141.21, 148.22, 155.59; MS (E1) m/z (rel. intensity) 251 (M⁺, 48), 236 (35), 220 (14), 206 (20), 191 (24), 178 (base), 156 (11), 128 (10), 110 (9), 101 (4), 75 (5), 73 (89), 53 (5) 43 (4). HRMS calculated for C₁₆H₁₇NSi 251.1130, found 251.1140; IR, (in CHCl₃, cm⁻¹) 2871, 2360, 1597, 1251, 1007, 911, 840, 741

### Example 8

### Synthesis of (Z)-2-But-1-en-3-ynyl-quinoline 8b

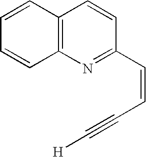

A solution of silylated quinoline 7b (0.020 g, 0.08 mmol) in 40 mL of methanol was cooled to 0° C. and then potassium carbonate (0.022 g, 0.16 mmol) was added to the mixture. The mixture was stirred for 1 hr at the indicated temperature, diluted with a saturated solution of sodium bicarbonate and extracted with hexanes. The organic layer was dried over sodium sulfate and concentrated on a rotary evaporator. The residue was purified by flash column chromatography on neutral alumina using 5:1 hexanes/ethyl acetate as the eluent to yield the product as a brown solid (0.134 g, 94%). ¹H NMR (300 MHz, CDCl₃) δ3.47 (1H, dd, J=0.9, 2.7 Hz), 6.08 (1H, dd, J=12.3, 2.7 Hz), 7.12 (1H, d, J=12.3 Hz), 7.54 (1H, td, J=6.9, 1.2 Hz), 7.68 (1H, td, J=6.9, 1.2 Hz), 7.80 (1H, d, J=8.4 Hz), 8.06 (1H, d, J=8.4 Hz), 8.16 (1H, d, J=9.0 Hz), 8.51 (1H, d, J=8.4 Hz); ¹³C NMR (75 MHz, CDCl₃) δ 81.50, 86.06, 111.48, 120.72, 127.12, 127.68, 127.76, 129.69, 129.93, 136.30, 142.01, 148.17, 155.23

### Example 9

### Synthesis of 3-Methoxymethyl indolizine 10a

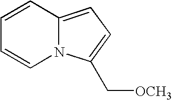

Potassium fluoride (0.058 g, 0.99 mmol) was added to a mixture of silylated enyne 7a (0.100 g, 0.49 mmol) in 20 mL of methanol. The mixture was heated under reflux for 1 hr and then allowed to cool to room temperature. The solvent was removed under reduced pressure and the residue dissolved in hexanes and washed with water (3×30 mL). The organic layer was dried over magnesium sulfate under nitrogen and evaporated to yield the pure product (0.072 g, 90%) as yellow oil. ¹H NMR (300 MHz, CDCl₃) δ 3.30 (3H, s), δ 4.74 (2H, s), δ 6.43 (1H, d, J=3.9 Hz), δ 6.56 (1H, td, J=6.0, 1.2 Hz), δ 6.77 (2H, m), δ 7.41 (1H, d J=9.0 Hz), δ 8.04 (1H, dd J=7.2, 0.9 Hz) ¹³C NMR (75 MHz, CDCl₃) δ 57.2, 66.1, 98.3, 110.6, 115.7, 117.5, 119.9, 122.1, 123.5, 134.3

### Example 10

### Synthesis of 3-Ethoxymethyl indolizine 10b

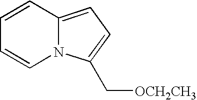

Compound 10b was synthesized analogously to compound 10a from compound 7a (0.100 g, 0.49 mmol,) and potassium fluoride (0.058 g, 0.99 mmol) in 20 mL of ethanol. The pure product was obtained after evaporation of solvent (0.080 g, 92%) as a yellow oil. ¹H NMR (300 MHz, CDCl₃) δ 1.21 (3H, t, J=6.9 Hz), δ 3.50 (2H, q, J=6.9 Hz), δ 4.77 (2H, s), δ 6.38 (1H, d, J=3.9 Hz), δ 6.55 (1H, td, J=6.9, 1.2 Hz), δ 6.75 (2H, m), δ 7.39 (1H, dt J=6.9, 1.2 Hz), δ 8.05 (1H, dd J=7.2, 1.2 Hz) ¹³C NMR (75 MHz, CDCl₃) δ 15.4, 64.3, 64.9, 98.2, 110.5, 115.3, 117.3, 119.5, 120.4, 123.5, 134.2; MS (EI) m/z (rel. intensity) 175 (M+, 20), 130 (base), 117 (5), 103 (3), 90 (3), 78 (5) 51 (3). HRMS calculated for C₁₁H₁₃NO 175.0997, found 175.0993; IR neat (cm⁻¹) 2975, 2870, 2340, 1630, 1503, 1360, 1315, 1245, 1158, 1087, 753

### Example 11

### Synthesis of 3-Allyloxymethyl-indolizine 10c

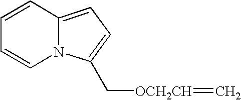

Compound 10c was synthesized analogously to compound 10a from compound 7a (0.100 g, 0.49 mmol) and potassium fluoride (0.058 g, 0.99 mmol) in 20 mL of allyl alcohol. The pure product was obtained after evaporation of solvent (0.093 g, 100%) as a yellow oil. ¹H NMR (300 MHz, CDCl₃) δ 3.98 (2H, td, J=2.7, 1.5 Hz), δ 4.82 (2H, s), δ 5.34 (2H, m), δ 5.98, (1H, m), δ 6.41 (1H, d, J=3.9 Hz), δ 6.58 (1H, td, J=6.9, 1.2 Hz), δ 6.79 (2H, m), δ 7.42 (1H, d, J=8.1 Hz), δ 8.09 (1H, dd, J=7.2, 1.2 Hz); ¹³C (75 MHz, CDCl₃) δ 63.6, 70.2, 98.3, 110.6, 115.6, 117.5, 117.7, 119.4, 119.9, 123.5, 134.3, 134.9; IR neat (cm⁻¹) 3079, 2901, 2340, 1631, 1537, 1502, 1360, 1204, 1054, 926, 818, 753

### Example 12

### Synthesis of 3-Isopropoxymethyl-indolizine 10d

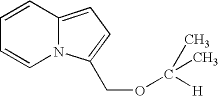

Compound 10d was synthesized analogously to compound 10a from compound 7a (0.100 g, 0.49 mmol) and potassium fluoride (0.058 g, 0.99 mmol) in 20 mL of 2-propanol. The pure product was obtained after evaporation of solvent (0.088 g, 94%) as a yellow oil. ¹H NMR (300 MHz, CDCl₃) δ 1.21 (9H, d, J=2.4 Hz), δ 3.67 (1H, m), δ 4.80 (2H, s), δ 6.41 (1H, d, J=3.6 Hz), δ 6.55 (1H, td, J=6.0, 1.2 Hz), δ 6.77 (2H, m), δ 7.41 (1H, d J=9.0 Hz), δ 8.08 (1H, d J=7.2 Hz); ¹³C NMR (75 MHz, CDCl₃) δ 22.3, 61.9, 69.9, 98.2, 110.5, 115.1, 117.3, 119.4, 120.63, 123.6, 134.2; IR: neat (cm⁻¹) 3087, 2977, 2340, 1632, 1504, 1315, 1245, 1118, 1040, 919, 816, 754; MS (EI) m/z (rel. intensity) 189 (M+, 20), 130 (base), 117 (4), 103 (4), 78 (5), 40 (3). HRMS calculated for C₁₂H₁₅NO 189.1148 found 189.1154.

### Example 13

### Synthesis of 3-tert-Butoxymethyl-indolizine 10e

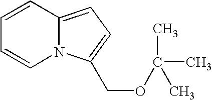

Compound 10e was synthesized analogous to compound 10a from compound 7a (0.100 g, 0.49 mmol) and potassium fluoride (0.058 g, 0.99 mmol) in 20 mL of tert-butanol. The pure product was obtained after evaporation of solvent (0.085 g, 84%) as a yellow solid. M.P (53-54° C.); ¹H NMR (300 MHz, CDCl₃) δ 1.34 (9H, s), δ 4.73 (2H, s), δ 6.39(1H, d, J=3.9 Hz), δ 6.53(1H, td, J=6.0, 1.2 Hz), δ 6.75(2H, m), δ 7.39 (1H, d J=9.0 Hz), δ 8.04 (1H, d J=6.9 Hz); ¹³C NMR (75 MHz, CDCl₃) δ 27.9, 56.5, 73.6, 98.3, 110.3, 114.5, 117.0, 119.4, 121.3, 123.4, 134.1; IR (cm⁻¹) 2978, 1633, 1364, 1316, 1192, 1047, 909, 740; IR in CHCl₃ (cm⁻¹) 2340, 1636, 1362, 1190, 1047, 911, 740

### Example 14

### Synthesis of 3-Pentyloxymethyl-indolizine 10f

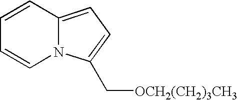 To a mixture of protected enyne 7a (0.060 g, 0.30 mmol) in 20 mL of pentanol was added cesium fluoride (0.068 g, 0.45 mmol). The mixture was head to reflux for 1 hr and cooled to room temperature. The mixture was diluted with a 40 mL solution of 1:1 methanol and water and extracted with hexanes (3×20 mL). The hexanes layer was washed with water (2×30 mL), dried over magnesium sulfate under nitrogen and concentrated. The residue was purified by flash column chromatography on basic alumina using petroleum ether as eluent to yield 0.020 g (31%) of the product as yellow oil. ¹H NMR (500 MHz, CDCl₃) δ 0.86 (3H, t, J=7.0 Hz), δ 1.29 (4H, m), δ 1. 56 (2H, m), δ 3.39 (2H, t, J=6.5 Hz), δ 4.78 (2H, s), δ 6.37 (1H, d, J=4.0 Hz), δ 6.53 (1H, td, J=6.5, 1.0 Hz), δ 6.74 (2H, m), δ 7.38 (1H, td J=6.5, 1.0 Hz), δ 8.03 (1H, dd J=7.0, 1.0 Hz); ¹³C NMR (125 MHz, CDCl₃) δ 14.2, 22.7, 28.6, 29.6, 64.5, 69.6, 98.1, 110.5, 115.3, 117.3, 119.4, 120.4, 123.5, 134.2; IR (in CHCl₃, cm⁻¹) 2913, 2848, 2242, 1462, 1316, 1089, 911, 731

### Example 15

### Synthesis of 3-Decyloxymethyl-indolizine 10 g

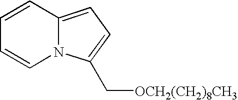

While stirring, cesium fluoride (0.079 g, 0.52 mmol), and decyl alcohol (0.600 g, 10.8 mmol) were added to a solution of enyne 7a (0.070 g, 0.35 mmol) in 20 mL of dry toluene. The mixture was refluxed for 24 hrs, allowed to cool to room temperature and concentrated. The residue was purified by flash column chromatography on basic alumina to yield the product as a yellow oil.

### Example 16

### Synthesis of 3-Cyclohexyloxymethyl-indolizine 10h

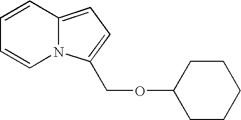

Compound 10h was synthesized analogously to compound 10f from compound 7a (0.070 g, 0.35 mmol) and cesium fluoride (0.079 g, 0.52 mmol) in 20 mL of cyclohexanol. The crude was purified by flash column chromatography (silica gel, 15:1 hexanes/ethyl acetate) to yield; 0.054 g, (68%) as yellow oil. The column was packed with hexanes and 3% triethylamine. ¹H NMR (300 MHz, CDCl₃) δ 1.33 (4H, m), δ 1.59 (2H, m), δ 1.73 (2H, m), δ 1.92 (2H, m), δ 3.34 (1H, m), δ 4.81 (2H, s), δ 6.38 (1H, d, J=3.6 Hz), δ 6.55 (1H, td, J=6.3, 1.2 Hz), δ 6.74 (2H, m), δ 7.36 (1H, d J=9.0 Hz), δ 8.07 (1H, d J=7.2 Hz); ¹³C NMR (75 MHz, CDCl₃) δ 24.4, 26.0, 32.4, 61.7, 76.1, 98.2, 110.4, 114.9, 117.2, 119.3, 120.8, 123.6, 134.2; IR neat (cm⁻¹) 3087, 2951, 2831, 2658, 2360, 1894, 1631, 1538, 1447, 1362, 1258, 1156, 1083, 949, 887, 7510

### Example 17

### Synthesis of 3-Benzyloxymethyl-indolizine 10i

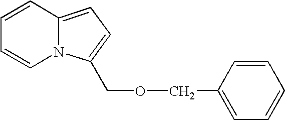 Compound 10i was synthesized analogously to compound 10g from compound 7a (0.070 g, 0.35 mmol), cesium fluoride (0.079 g, 0.52 mmol) and benzyl alcohol (0.376 g, 3.5 mmol) in 20 mL of toluene for 2 hrs. The crude was purified by flash column chromatography on basic alumina to yield 0.068 g, (82%) as yellow oil ¹H NMR (300 MHz, CDCl₃) δ 4.47 (2H, s), δ 4.85 (2H, s), δ 6.42 (1H, d, J=3.9 Hz), δ 6.58 (1H, td, J=7.2, 1.5 Hz), δ 6.74 (1H, td, J=7.5, 1.5 Hz), δ 6.79 (1H, d, J=3.9 Hz), δ 7.43 (6H, m), δ 8.07 (1H, d J=7.2 Hz); ¹³C NMR (75 MHz, CDCl₃) δ 63.6, 71.2, 98.3, 110.6, 115.8, 117.5, 119.4, 119.9, 123.6, 127.2, 127.9, 128.3, 128.7, 128.8, 134.4, 138.3; IR (in CHCl₃, cm⁻¹) 3030, 2848, 2245, 1462, 1315, 1204, 1086, 911, 730

### Example 18

### Synthesis of 3-MethyoxymethylD-indolizine 10 j

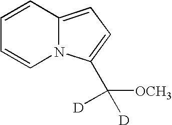

Potassium fluoride (0.040 g, 0.70 mmol) was added to a solution of the protected enyne 7a (0.070 g, 0.35 mmol) in 20 mL of single deuterium labeled methanol (CH₃OD). The mixture was refluxed for an hour and allowed to cool to room temperature. The mixture was diluted with 25 mL of water and extracted with petroleum ether (30 mL×3). The petroleum ether layer was dried over magnesium sulfate under nitrogen and concentrated to yield the pure product as yellow oil.

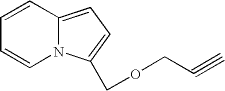

### Synthesis of 3-Prop-2-ynyloxymethyl-indolizine

To a solution of enyne-TMS 7a (0.070 g, 0.35 mmol) in 20 mL of anhydrous toluene were added cesium fluoride (0.079 g, 0.52 mmol) and propagyl alcohol (0.195 g, 3.5 mmol). The mixture was heated to reflux for 3 hrs, allowed to cool to room temperature and concentrated on a rotary evaporator. The residue was dissolved in 30 mL of a 1:1 solution of MeOH/H₂O and extracted with hexanes (2×25 mL). The organic layer was washed with water, dried over magnesium sulfate under nitrogen and concentrated. The residue was purified by column chromatography on basic alumina using hexanes and ethyl acetate (20:1) as eluent. Yield; 0.047 g, (73%) as a yellow oil. ¹H NMR (500 MHz, CDCl₃) δ 2.47 (1H, t, J=2.5 Hz), 4.07 (2H, d, J=2.5 Hz), δ 4.89 (2H, s), δ 6.39 (1H, d, J=3.5 Hz), δ 6.56 (1H, td, J=6.5, 1.5 Hz), δ 6.73 (1H, td, J=6.5, 1.5 Hz), δ 6.82(1H, d, J=4.0 Hz), δ 7.39(1H, d J=9.0 Hz), δ 8.07(1H, d J=7.0 Hz); ¹³C NMR(125 MHz, CDCl₃) δ56.2, 62.9, 74.9, 79.9, 98.5, 110.7, 116.3, 117.7, 118.9, 119.4, 123.5, 134.5.

### Example 19

### Synthesis of 1-Methoxymethyl-pyrrolo[1,2-a]quinoline 11a

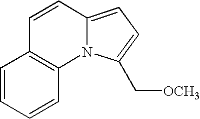

To a mixture of silylated enyne 7b (0.050 g, 0.20 mmol) in methanol (20 mL) was added potassium fluoride (0.023 g, 0.40 mmol). The mixture was refluxed for an hour and allowed to cool to room temperature. The solvent was removed on a rotary evaporator and the residue was dissolved in hexanes, washed three times with water (30 mL×3), and dried over sodium sulfate. Evaporation of the solvent under reduced pressure yielded the pure product (0.040 g, 95%) as a yellow oil. ¹H NMR (300 MHz, CDCl₃) δ 3.41 (3H, s), δ 4.87 (2H, s), δ 6.46 (1H, d, J=3.6 Hz), δ 6.74 (1H, d, J=3.9 Hz), δ 6.98 (1H, d, J=9.3 Hz), δ 7.32 (2H, m), δ 7.49 (1H, td, J=7.2, 1.5 Hz), δ 7.62 (1H, dd J=7.8, 1.8 Hz), δ 8.40 (1H, d, J=8.4 Hz); ¹³C NMR (75 MHz, CDCl₃) δ 56.8, 67.9, 102.0, 117.6, 118.0, 119.3, 119.8, 123.7, 125.3, 125.4, 127.9, 128.5, 134.1, 135.2; IR neat (cm⁻¹) 3050, 2817, 1694, 1607, 1555, 1479, 1321, 1220, 1123, 1081, 943, 897, 752.

### Example 20

### Synthesis of 1-Ethoxymethyl-pyrrolo[1,2-a]quinoline 11b

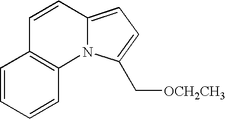

Compound 11b was synthesized analogously to compound 11a from compound 7b (0.050 g, 0.20 mmol) and potassium fluoride ((0.023 g, 0.40 mmol) in 20 mL of ethanol. The pure product was obtained after evaporation of solvent (0.043 g, 96%) ¹H NMR (300 MHz, CDCl₃) δ 1.29 (3H, t, J=7.2 Hz), δ 3.68 (2H, q, J=6.9 Hz), δ 4.94 (2H, s), δ 6.47 (1H, d, J=3.9 Hz), δ 6.75 (1H, d, J=3.9 Hz), δ 7.01 (1H, d, J=9.0 Hz), δ7.34(2H, m), δ 7.53(1H, td, J=7.5, 1.8 Hz), δ 7.64(1H, dd J=7.8, 1.5 Hz), δ 8.48 (1H, d, J=8.7 Hz) ¹³C NMR (75 MHz, CDCl₃) δ 15.4, 64.7, 66.2, 102.0, 117.7, 117.8, 119.3, 119.7, 123.7, 125.3, 125.9, 127.8, 128.5, 134.0, 135.3; MS (EI) m/x (rel. intensity) 225 (M⁺,80), 224 (2), 194 (5), 180 (base), 179 (24), 167 (9), 141 (6), 128 (4), 90 (3). HRMS calculated for C₁₅H₁₅NO 225.1154, found 225.1146; IR neat (cm⁻¹) 3050, 2976, 2795, 1608, 1555, 1321, 1124, 1081,

### Example 21

### Synthesis of 1-Allyloxymethyl-pyrrolo[1,2-a]quinoline 11c

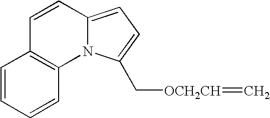

Compound 11c was synthesized analogously to compound 11a from compound 7b (0.050 g, 0.20 mmol) and potassium fluoride ((0.023 g, 0.40 mmol) in 20 mL of allyl alcohol. The pure product was obtained after evaporation of solvent (0.047 g, 100%) as a yellow oil. ¹H NMR (300 MHz, CDCl₃) δ 4.15 (2H, td, J=2.7, 1.2 Hz), δ 4.99 (2H, s), δ 5.39 (2H, m), δ 6.04, (1H, m), δ 6.49 (1H, d, J=3.6 Hz), δ 6.77 (1H, d, J=3.6 Hz), δ 7.03 (1H, d, J=9.3 Hz), δ 7.36 (2H, m), δ 7.55 (1H, td J=7.2, 1.8 Hz), δ 7.66 (1H, dd J=7.8, 1.2 Hz), δ 8.54 (1H, d, J=8.4 Hz); ¹³C NMR (75 MHz, CDCl₃) δ 65.5, 69.9, 102.1, 117.7, 117.8, 118.03, 119.3, 119.8, 123.7, 125.3, 125.4, 127.9, 128.5, 134.1, 134.8, 135.2; IR neat (cm⁻¹) 3077, 2985, 2340, 1887, 1645, 1555, 1321, 1220, 1057, 994, 861, 798, 752

### Example 22

### Synthesis of 1-Isopropoxyymethyl-pyrrolo[1,2-a]quinoline 11d

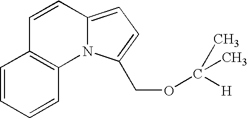

Compound 11d was synthesized analogously to compound 11a from compound 7b (0.050 g, 0.20 mmol) and potassium fluoride (0.023 g, 0.40 mmol) in 20 mL of 2-propanol. The pure product was obtained after evaporation of solvent (0.041 g, 86%) as a yellow oil. ¹H NMR (300 MHz, CDCl₃) δ 1.22 (6H, d, J=1.2 Hz), δ 3.87 (1H, m), δ 4.96 (2H, s), δ 6.48 (1H, d, J=3.9 Hz), δ 6.75 (1H, d, J=3.6 Hz), δ 7.00 (1H, d, J=9.3 Hz), δ7.34 (2H, m), δ 7.52 (1H, td J=7.2, 1.5 Hz), δ 7.64 (1H, dd J=7.8, 1.5 Hz), δ 8.56 (1H, d, J=8.4 Hz) ¹³C NMR (75 MHz, CDCl₃) δ 22.4, 63.9, 70.0, 102.1, 117.5, 118.0, 119.3, 119.7, 123.7, 125.3, 126.2, 127.7, 128.4, 134.0, 135.3; MS (EI) m/z (rel. intensity) 239 (M⁺, 45), 196(5), 180 (base), 179 (16), 168 (10), 152 (3), 128 (3). HRMS calculated for C₁₆H₁₇NO 239.1302, found 239.1302; IR neat (cm⁻¹) 3051, 2973, 2871, 2359, 1608, 1555, 1367, 1124, 1045

### Example 23

### Synthesis of 1-tert-Butoxymethyl-pyrrolo[1,2-a]quinoline 11e

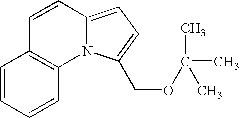

Compound 11e was synthesized analogously to compound 11a from compound 7b (0.050 g, 0.20 mmol) and potassium fluoride ((0.023 g, 0.40 mmol) in 20 mL of tert-butanol. The pure product was obtained after evaporation of solvent (0.052 g, 97%) as a yellow solid. M.P. (43-45° C.); ¹H NMR (300 MHz, CDCl₃) δ 1.32 (9H, s), δ 4.81 (2H, s), δ 6.40 (1H, d, J=3.9 Hz), δ 6.66 (1H, d, J=3.6 Hz), δ 6.90 (1H, d, J=9.3 Hz), δ 7.26 (2H, m), δ 7.43 (1H, td J=7.2, 1.5 Hz), δ 7.56 (1H, d, J=1.8 Hz), δ 8.53 (1H, d, J=8.7 Hz); ¹³C NMR (75 MHz, CDCl₃) δ 28.1, 58.5, 74.14, 102.3, 116.8, 118.1, 119.4, 119.4, 123.6, 125.3, 127.1, 127.3, 128.4, 133.7, 135.2

### Example 24

### Synthesis of 1-Pentyloxymethyl-pyrrolo[1,2-a]quinoline 11f

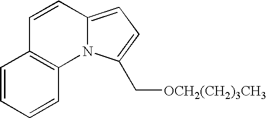

To a mixture of protected enyne 7b (0.050 g, 0.20 mmol) in 20 mL of pentanol was added cesium fluoride (0.045 g, 0.30 mmol). The mixture was head to reflux for 1 hr and cooled to room temperature. The mixture was poured into a 40 mL solution of 1:1 methanol and water and extracted with hexanes (3×20 mL). The hexanes layer was washed with water (2×30 mL), dried over sodium sulfate and concentrated. The residue was purified by flash column chromatography (silica gel, 15:1 hexanes/ethyl acetate). Yield; 0.030 g, (57%) as a yellow oil. ¹H NMR (300 MHz, CDCl₃) δ 0.88 (3H, t, J=7.5 Hz), δ 1.36 (4H, m), δ 1.65 (2H, m), δ 3.61 (2H, t, J=6.6 Hz), δ 4.94 (2H, s), δ 6.49 (1H, d, J=3.6 Hz), δ 6.77 (1H, d, J=3.9 Hz), δ 7.02 (1H, d, J=9.0 Hz), δ 7.36 (2H, m), δ 7.51 (1H, td, J=7.8, 1.5 Hz), δ 7.66 (1H, dd J=7.8, 1.5 Hz), δ 8.50 (1H, d, J=9.0 Hz); ¹³C NMR (75 MHz, CDCl₃) δ 14.2, 22.7, 28.8, 29.6, 66.4, 69.5, 102.0, 117.7, 117.9, 119.3, 119.7, 123.7, 125.3, 125.9, 127.8, 128.4, 133.9, 135.2; IR in CHCl₃ (cm⁻¹) 2871, 1631, 1478, 1424, 1322, 1123, 1085, 908, 876, 799, 737

### Example 25

### Synthesis of 1-Decyloxymethyl-pyrrolo[1,2-a]quinoline 11g

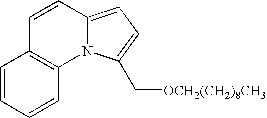

While stirring, cesium fluoride (0.045 g, 0.30 mmol), and decyl alcohol (0.630 g, 4.0 mmol) were added to a solution of enyne 7b (0.050 g, 0.020 mmol) in 20 mL of dry toluene. The mixture was refluxed for 24 hrs, allowed to cool to room temperature and concentrated. The residue was purified by flash column chromatography (silica gel, hexanes) to yield, 0.041 g, (61%) as a yellow oil. ¹H NMR (300 MHz, CDCl₃) δ 0.88 (3H, t, J=3.3 Hz), δ 1.33 (14H, m), δ 1.62 (2H, m), δ 3.59 (1H, t, J=6.6 Hz), 4.92 (2H, s), δ 6.47 (1H, d, J=3.9 Hz), δ 6.74 (1H, d, J=3.9 Hz), δ 7.00 (1H, d, J=9.3 Hz), δ 7.34 (2H, m), δ 7.49 (1H, td J=7.2, 1.5 Hz), δ 7.63 (1H, dd J=8.2, 1.5 Hz), δ 8.48 (1H, d, J=8.4 Hz);

### Example 26

### Synthesis of 1-Cyclohexyloxymethyl-pyrrolo[1,2-a]quinoline 11h

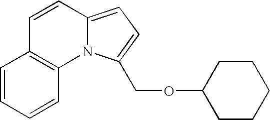

Compound 11h was synthesized analogously to compound 11f from compound 7b (0.050 g, 0.20 mmol) and cesium fluoride (0.045 g, 0.29 mmol) in 20 mL of cyclohexanol. The crude was purified by flash column chromatography (silica gel, 15:1 hexanes/ethyl acetate) to yield; 0.043 g, (77%) as yellow oil. ¹H NMR (300 MHz, CDCl₃) δ 1.25 (3H, m), δ 1.38 (2H, m), 1.54 (1H, m), δ 1.78 (2H, m), δ 1.99 (2H, m), δ 3.56 (1H, m), 4.98 (2H, s), δ 6.47 (1H, d, J=3.6 Hz), δ 6.74 (1H, d, J=3.9 Hz), δ 6.99 (1H, d, J=9.3 Hz), δ 7.33 (2H, m), δ 7.52 (1H, td, J=7.2, 1.5 Hz), δ 7.64 (1H, dd J=7.2, 1.5 Hz), δ 8.59 (1H, d, J=9.0 Hz); ¹³C NMR (75 MHz, CDCl₃) δ 24.5, 26.1, 32.5, 63.5, 76.1, 102.1, 117.5, 118.1, 119.3, 119.6, 123.6, 125.3, 126.4, 127.6, 128.4, 133.9, 135.2; IR neat (cm⁻¹) 3049, 2833, 1607, 1553, 1424, 1360, 1220, 1122, 1071, 949, 875, 796

### Example 27

### Synthesis of 1-Benzyloxymethyl-pyrrolo[1,2-a]quinoline 11i

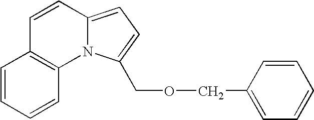

Compound 11i was synthesized analogously to compound 11g from compound 7b (0.030 g, 0.12 mmol) and cesium fluoride (0.027 g, 0.18 mmol), and benzyl alcohol (0.129 g, 1.2 mmol). The residue was purified by flash column chromatography (silica gel, 15:1 hexanes/ethyl acetate) to yield, 0.016 g, (47%) as a yellow oil. ¹H NMR (300 MHz, CDCl₃) δ4.64 (2H, s), δ 5.01 (2H, s), δ 6.48 (1H, d, J=3.9 Hz), δ 6.75 (1H, d, J=3.9 Hz), δ 7.02 (1H, d, J=9.3 Hz), δ 7.46 (7H, m), δ 7.49 (1H, td, J=7.5, 1.8 Hz), δ 7.65 (1H, dd J=7.8, 1.5 Hz), δ 8.53 (1H, d, J=8.7 Hz); ¹³C NMR (75 MHz, CDCl₃) δ 65.6, 71.1, 102.1, 117.8, 118.2, 119.3, 119.9, 123.7, 125.3, 125.4, 127.9, 127.9, 128.4, 127.5, 128.7, 134.2, 135.2, 138.2; IR (in CHCl₃, cm⁻¹) 3030, 2848, 2245, 1731, 1606, 1555, 1472, 1309, 1123, 1061, 910, 730

### Example 28

### Synthesis of 1-MethoxymethylD-pyrrolo[1,2-a]quinoline 11j

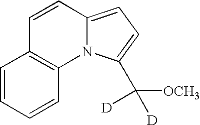

Potassium fluoride (0.037 g, 0.636 mmol) was added to a solution of the protected enyne 7b (0.080 g, 0.32 mmol) in 20 mL of single deuterium labeled methanol (CH₃OD). The mixture was refluxed for an hour and allowed to cool to room temperature. The mixture was diluted with 25 mL of water and extracted with petroleum ether (30 mL×3). The petroleum ether layer was dried over sodium sulfate and concentrated to yield the pure product (0.063 g, 87%) as a yellow oil. ¹H NMR (300 MHz, CDCl₃) δ 3.46 (3H, s), δ 6.51 (1H, d, J=3.9 Hz), δ 6.79 (1H, d, J=3.9 Hz), δ 7.03 (1H, d, J=9.3 Hz), δ 7.37 (2H, m), δ 7.54 (1H, td J=7.2, 1.5 Hz), δ 7.66 (1H, dd J=7.5, 1.2 Hz), δ 8.45 (1H, d, J=8.7 Hz) ¹³C NMR (75 MHz, CDCl₃) δ 56.7, 67.6 (m), 102.1, 117.6, 118.0, 119.3, 119.8, 123.8, 125.3, 125.4, 127.9, 128.5, 134.1, 135.2; IR neat (cm⁻¹) 3051, 2986, 2341, 2142, 2080, 1940, 1608, 1552, 1505, 1383, 1223, 1095, 973, 920, 846, 752

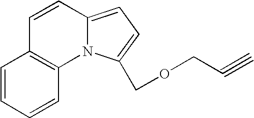

### Synthesis of 1-Prop-2-ynyloxymethyl-pyrrolo[1,2-a]quinoline

To a solution of enyne-TMS 7b (0.050 g, 0.20 mmol) in 20 mL of anhydrous toluene were added cesium fluoride (0.045 g, 0.29 mmol) and propagyl alcohol (0.112 g, 2.0 mmol). The mixture was heated to reflux for 3 hrs, allowed to cool to room temperature and concentrated on a rotary evaporator. The residue was dissolved in 30 mL of a 1:1 solution of MeOH/H₂O and extracted with hexanes (2×25 mL). The organic layer was washed with water, dried over magnesium sulfate and concentrated. Final purification was achieved by preparatory thin layer chromatography. Yield; 0.024 g (51%) as a yellow oil. ¹H NMR (500 MHz, CDCl₃) δ 2.52 (1H, t, J=2.5 Hz), 4.25 (2H, d, J=2.5 Hz), δ 5.09 (2H, s), δ 6.49 (1H, d, J=3.5 Hz), δ 6.82 (1H, d, J=4.0 Hz), δ 7.02(1H, d, J=9.0 Hz), δ 7.34(2H, m), δ 7.52(1H, td, J=7.0, 1.5 Hz), δ 7.65(1H, dd J=8.0, 2.0 Hz), δ 8.49 (1H, d, J=8.5 Hz); ¹³C NMR (75 MHz, CDCl₃) δ 55.9, 64.8, 75.1, 79.8, 102.2, 117.8, 118.8, 119.3, 120.1, 123.8, 124.3, 125.3, 128.0, 128.6, 134.4, 135.1; IR (neat, cm⁻¹) 3292, 2848, 2248, 2116, 1732, 1608, 1472, 1322, 1123, 1063, 912, 800, 730

### REFERENCES

1. (a) Comprehensive Heterocyclic Chemistry. The Structure, Reactions, Synthesis, and Uses of Heterocyclic Compounds, Eds. Katritzky, A. R. and Rees, C. W., Vols 1-8, Pergamon Press, Oxford, 1984
2. Flitsch, W. In Comprehensive Heterocyclic Chemistry, edited by A. R. Katrizky and C. W. Rees, Vol 4, p 443, Pergamon Press, Oxford, 1984. (c) Swinborne, J. H. Hunt and G. Klinkert. Adv. Heterocycl. Chem. 23, 103-170, 1978
3. For a review, see (a) Uchida, T.; Matsumoto, K. Syntheisis, 1976, 209 (b) Katritzky, A. R.; Qui, G.; Yang, B.; He, H. Y. J. Org. Chem. 1999, 64, 7618-7621 (c) Zhang, X. C.; Huang, W. Synthesis, 1999, 51-54
4. For recent reviews see: (a) Micheal, J. P. Alkaloids, 2001, 55, 91 (b) Micheal, J. P. Nat. Prod. Rep. 2002, 19, 742.
5. Castanospermine: (a) Hohenschultz, L. D.; Bell, E. A.; Jewess, P. J.; Leworthy, D. P.; Pryce, R. J.; Arnold, E.; Clardy, J. Phytochemistry 1981, 20, 811. (b) Nash, R. J.; Fellows, L. E.; Dring, J. V.; Striton, C. H., Carter, D.; Hegarty, M. P.; Bell, E. A. Phytochemistry, 1988, 27, 1403.
6. Swainsonine: (a) Guengerich, F. P.; DiMari, S. J.; Broquist, H. P. J. Am. Chem. Soc. 1973, 95, 2055. (b) Colegate, S. M.; Dorling, P. R.; Huxtable, C. R. Aust. J. Chem. 1979, 32, 2257. (c) Molyneux, R. J.; James, L. F. Science, 1982, 216, 190. (d) Davis, D.; Schwaru, P.; Hernandez, T.; Mitchell, M.; Warnock, B.; Elbein, A. D. Plant Physiol. 1984, 76, 972. (e) Yasuda, N.; Tsutsumi, H.; Takaya, T. Chem. Lett. 1984, 1201.
7. Heterocyclic systems with bridgehead nitrogen atom (Part 1) in The Chemistry of Heterocyclic Compounds, Vol 15, Ed. Mosby, W. L. Series Ed. Weissberger, A., Wiley-interscience, 1961.
8. (a) Gubin, J.; Vogelaer, H.; Inion, H.; Houben, C.; Lucchetti, J.; Mahaux, J.; Rosseels, G.; Peiren, M.; Clinet, M.; Polster, P.; Chatelain, P. J. Med. Chem.; 1993, 36, 1425 (b) Gupta, S. P.; Mathur, A. N.; Nagappa, A. N.; Kumar, D.; Kumaran, S. Eur. J. Med. Chem., 2003, 38, 867-873 (c) Gundersen, L. L.; Malterud, K. E.; Negussie, A. H.; Rise, F.; Teklu, S.; Østby, O. B. Bioorg. Med. Chem. 2003, 11, 5409-5415
9 (a) Rise, F.; Wikström, H.; Ugland, S. Dijkstra, D.; Gunersen, L.-L.; P. De Boer, A.; Bast. I.; Haenen, G.; Antosen, φ.; Liao, Y.; Nasir, A. I. PCT Int. Appl. WO 9621, 662/1996; Chem. Abstr. 1996, 125, 195681 (b) Nasir, A.; Gunersen, L.-L.; Rise, F.; Antosen, φ.; Kristensen, T.; Langhelle, B.; Bast, A.; Custers, I.; Haenen, G. R. M. M.; Wikström, H. Bioorg. Med. Chem. Lett. 1998, 32,1829-1832. (c) Østby, O. B. Dalhus, B.; Gundersen, L.-L.; Rise, L.; Bast, F.; Haenen, G. R. M. M. Eur. J. Org. Chem, 2000, 3763-3770.
10. (a) Scholtz, M. Ber. Dtsch. Chem. Ges. 1912, 45, 734 (b) Boekelheide, V.; Windgassen, R. J. J. Chem. Soc. 1959, 81, 1456
(11) Tschitschibabin, A. E. Ber. Dtsch. Chem. Ges. 1927, 60, 1607 (d) Jones, G.; Stanyer, J. J. Chem. Soc. 1969, 901
12. (a) Padwa, A.; Austin, D. J.; Precedo, L.; Zhi, L. J. Org. Chem. 1993, 58, 1144 (b) Wei, X.; Hu, Y.; Li, t.; Hu, H. J. Chem. Soc., Perkin Trans. 1 1993, 2487 (c) Siriwardana, A. I.; Nakamura, I.; Yamamoto, Y. J. Org. Chem, 2004, 69 (9) 3202-3204 (d) Acheson, R. M.; Robinson, D. A. J. Chem. Soc. 1968, 1633 (e) Fang, X.; Wu, Y. M.; Deng, J.; Wang, S. W. Tetrahedron, 2004, 60 (25), 5487-5493
13. Matsumoto, M; Kuroda, K. Tetrahedron Lett. 1980, 4021-4024
14. Takahashi, S.; Kuroyama, Y.; Sonogashira, K.; Higihara, N. Synthesis, 1980, 627-630

The foregoing is illustrative of the present invention, and is not to be construed as limiting thereof. The invention is defined by the following claims, with equivalents of the claims to be included therein.

That which is claimed is:

1. A method of making a compound of Formula I: 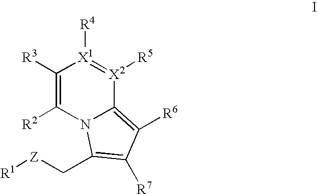
wherein:
X¹ and X² are each independently N or C, subject to the provisos that R⁴ is absent when X¹ is N and R⁵ is absent when X² is N;
Z is O or S;
R¹ is selected from the group consisting of H, alkyl, alkenyl, alkynyl, aryl, arylalkyl, alkoxyalkyl, alkylthioalkyl, aryloxyalkyl, alkenyloxyalkyl, silyl, trialkylsilyl, siloxyalkyl, tetrahydropyranyl, tetrahydrothiopyranyl, 1,4-Dioxan-2-yl, tetrahydrofuranyl, tetrahydrothiofurnyl, benzyl, p-(methylsulfinyl) benzyl, 2-picoyl, 4-picoyl, 2-quinolinylmethyl, 1-pyrenylmethyl-, 9-(9-phenyl)xanthenyl-, naphthanyl-, cyclodextrins, carboranes, halo, and solid supports;
R², R³, R⁴, R⁵, R⁶ and R⁷ are each independently selected from the group consisting of H, akyl, halo, alkenyl, alkoxy, alkoxyalkyl, alkoxycarbonyl, alkoxycarbonylalkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, haloalkyl, hydroxy, hydroxyalkyl, mercapto, and nitro;
or R² and R³ together form a group of the formula: 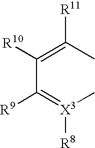
wherein:
X³ is N or C, subject to the proviso that R⁸ is absent when X³ is N;
R⁸, R⁹, R¹⁰, and R¹¹ are each independently selected from the group consisting of H, halo, alkyl, alkenyl, alkoxy, alkoxyalkyl, alkoxycarbonyl, alkoxycarbonylalkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, haloalkyl, hydroxy, hydroxyalkyl, mercapto, and nitro;
said method comprising reacting a compound of Formula II 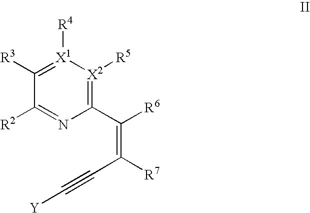
wherein Y is H, alkyl, alkenyl, aryl, or trialkylsilyl and R², R³, R⁴, R⁵, R⁶ and R⁷ are as given above,
with a compound selected from the group consisting of R¹OH and R¹SH, where R¹ is as given above, in the presence of a base to produce said compound of Formula I.

2. The method of claim 1, wherein said base is KF or CsF.

3. The method of claim 1, wherein said reaction is carried out at a temperature of 30 to 150° C.

4. The method of claim 1, wherein said reaction is carried under reflux conditions.

5. The method of claim 1, wherein said compound of Formula II is produced by reacting a compound of Formula III: 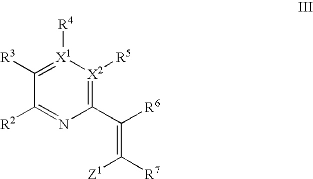
where Z¹ is halo and R², R³, R⁴, R⁵, R⁶ and R⁷ are as given above,
with (trialkylsilyl)acetylene in the presence of a base and a transition metal complex to produce a compound of Formula II.

6. The method of claim 5, wherein said (trialkylsilyl)acetylene is (trimethylsilyl)acetylene.

7. The method of claim 4, wherein said base is triethylamine.

8. The method of claim 4, wherein said transition metal complex is a palladium complex.

9. The method of claim 1, wherein:
R² and R³ together form a group of the formula: 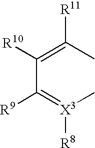
said compound of Formula II has the Formula IIa: 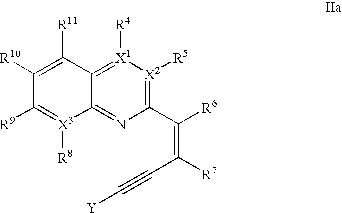
and X¹, X², X³, Y, R⁴, R⁵, R⁶, R⁷, R⁸, R⁹, R¹⁰, and R¹¹ are as given above.

10. The method of claim 1, wherein R⁷ is not H.

11. A compound of Formula Ia: 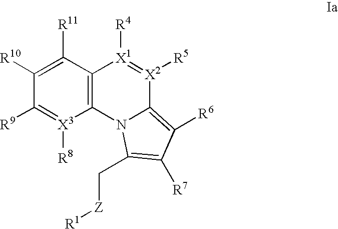
wherein:
X¹, X² and X³ are each independently N or C, subject to the provisos that R⁴ is absent when X¹ is N; R⁵ is absent when X² is N, and R⁸ is absent when X³ is N;
Z is O or S;
R¹ is selected from the group consisting of H, alkyl, alkenyl, alkynyl, aryl, arylalkyl, alkoxyalkyl alkylthioalkylaryloxyalkyl; alkenyloxyalkyl; silyl, trialkylsilyl, siloxyalkyl, tetrahydropyranyl, tetrahydrothiopyranyl, 1,4-Dioxan-2-yl, tetrahydrofuranyl, tetrahydrothiofurnyl, benzyl, p-(methylsulfinyl) benzyl, 2-picoyl, 4-picoyl, 2-quinolinylmethyl, 1-pyrenylmethyl-, 9-(9-phenyl)xanthenyl-, naphthanyl-cyclodextrins, carboranes, halo, and solid supports;
R², R³, R⁴, R⁵, R⁶, R⁷, R⁸, R⁹, R¹⁰, and R¹¹ are each independently selected from the group consisting of H, akyl, halo, alkenyl, alkoxy, alkoxyalkyl, alkoxycarbonyl, alkoxycarbonylalkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, haloalkyl, hydroxy, hydroxyalkyl, mercapto, and nitro;
and salts thereof.

12. The compound of claim 11, subject to the proviso that R⁷ is not H.

13. A compound of the formula IIa: 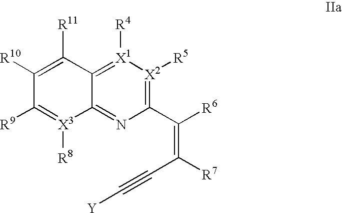
wherein:
X¹, X² and X³ are each independently N or C, subject to the provisos that R⁴ is absent when X¹ is N; R⁵ is absent when X² is N, and R⁸ is absent when X³ is N;
Y is H, alkyl, alkenyl, aryl, or trialkylsilyl; and
R², R³, R⁴, R⁵, R⁶, R⁷, R⁸, R⁹, R¹⁰, and R¹¹ are each independently selected from the group consisting of H, akyl, halo, alkenyl, alkoxy, alkoxyalkyl, alkoxycarbonyl, alkoxycarbonylalkyl, alkylcarbonyl, alkylcarbonylalkyl, alkylcarbonyloxy, alkylsulfonyl, alkylthio, alkynyl, aryl, arylalkoxy, arylalkyl, arylcarbonyl, aryloxy, carboxy, carboxyalkyl, cyano, cyanoalkyl, formyl, haloalkyl, hydroxy, hydroxyalkyl, mercapto, and nitro;
and salts thereof.

14. The compound of claim 13, subject to the proviso that R⁷ is not H.
